(19) United States
(12) Patent Application Publication
(10) Pub. No.: US 2026/0334764 A1
(43) Pub. Date: Oct. 29, 2026

(54) END ADAPTOR, CONTINUUM ROBOT, INSPECTION SYSTEM, AND METHODS FOR OPERATION OF SAME

(71) Applicant: CANON U.S.A., INC., Melville, NY (US)

(72) Inventor: Annie Yujin Edge, Somerville, MA (US)

(21) Appl. No.: 19/187,873

(22) Filed: Apr. 23, 2025

(51) Int. Cl. A61B 1/00; A61B 1/05

(52) U.S. Cl. CPC A61B 1/00154; A61B 1/05

(57) ABSTRACT

Disclosed are an end adaptor, a continuum robot, an inspection system, and methods for operation of same, with the end adaptor being arranged for insertion through a channel that extends through at least a part of the continuum robot, and including first and second ends with a body extending therebetween, and at least one protuberance extending from the body in a transverse direction substantially parallel to a longitudinal direction. The at least one protuberance includes a distal and proximal ends, and a center extending therebetween. The second end is arranged to abut a shaft for moving the end adaptor in the channel. Upon moving the end adaptor to a distal end of the channel, the first end aligns with a tip of the continuum robot, without the first end protruding from the distal end.

### BACKGROUND

### Field of the Disclosure

The present disclosure relates generally to medical devices and, more particularly, to devices and methods for image orientation during navigation of a continuum robot to guide tools and instruments, such as probes, endoscopes and catheters.

### Description of Related Art

Endoscopy is a medical procedure allowing remote inspection, manipulation, and/or treatment of internal organs using flexile devices inserted through natural orifices. The complexity of anatomical pathways in a patient, the limited flexibility of existing instruments, and the need to use multiple instruments may limit such procedures. Continuum robots are generally composed of a passive proximal section and an active distal section. The proximal passive section can be rigid, semi-rigid, or flexible. The active distal section includes a steerable tip that is remotely actuated by control wires connected to actuation wheels located on the handle of the device. Continuum robots may be equipped with an imaging device, e.g., a chip on tip (COT) camera, a light source, irrigation and/or suction channels, and at least one instrument channel for passing interventional tools. Endoscopic instruments may have at least three degrees of freedom (3DOF) for insertion, rotation and grasping operations. However, confined workspaces, limited visibility, and varied orientation of targets may limit usability. For example, four hands may be required to manually control a continuum robot and associated instruments during certain procedures. To that end, an operator may need to master a combination of accurate tip angulations, shaft management, and instrument insertion procedures, while communicating with an assistant to actuate the instrument and hold the endoscopic shaft in a correct position. Techniques have been proposed and robotic continuum robot systems have been developed to alleviate the complexity of such procedures.

In minimally invasive medical procedures, certain techniques may use flexible tubular instruments, e.g., a catheter, inserted through natural orifices or surgical incisions to deliver imaging tools and/or diagnostic tools, e.g., forceps, brush, and/or needle, or therapeutic tools, e.g., an ablation probe, to reach a target location. The continuum robot may contain driving wires and/or tendons to control its shape and tip orientation in a continuous curving structure. When mechanically advanced and actuated, the catheter may constitute a continuum robot.

To assist a user navigating to the target through natural passageways, e.g., airways, or through surgically created passageways, anatomical models are prepared using pre-operative or intra-operative imaging, e.g., computed tomography (CT) and/or magnetic resonance imaging (MRI). Thus, target tissue and a planned path may be identified and navigation can be virtually simulated. Use of three-dimensional (3D) tracking technologies, e.g., electromagnetic and/or shape sensing, with sensors embedded in the catheter may allow for real-time localization in the registered anatomical model during a procedure.

Navigation of the catheter may be guided by an imaging device, e.g. camera, videoscope or the like, arranged at the distal tip of the catheter shaft. A display device, such as a liquid crystal display (LDC) monitor provided in a console or attached to a wall, may display an image within a field of view (FOV) of the imaging device to assist navigation to a target site. The orientation of the FOV, the controller, and the catheter may be mapped (calibrated) before inserting the catheter, e.g., inserting into a patient's body. As the user manipulates the continuum robot to obtain a real time view, the FOV image from the imaging device is transferred to the display device. Ideally, the displayed FOV image allows the user to relate to the endoscopic image as if the user's own eyes were actually inside the cavity. Thus, an expectation exists of matching of image orientation (roll) of real and virtual views. Accordingly, when controlling robot actuation, a direction of motion with respect to the image view display orientation may be predicted. For example, if the catheter tip is commanded to move up, anatomy that is observed in the image FOV is expected to shift downwards, and vice versa.

During navigation, the camera may rotate or roll relative to the distal tip of the catheter, resulting in errors in the correlation of steering inputs to visual guidance. Techniques for controlling rotation of the displayed image include measuring an orientation of an endoscope and then rotating the endoscopic image optically, mechanically, or electronically to compensate for the change in orientation of the endoscope tip or rotation of the camera. See, US 2011/0026787, US 2013/0204126, U.S. Pat. Nos. 8,864,652, 10,275,899, 10,376,134, 10,987,179.

U.S. Pat. No. 10,588,597 to Zhao discusses methods for deploying an interventional instrument using a tracked catheter with an imaging probe that extends beyond the catheter tip, including determining the roll angle between the image frame of reference and catheter frame of reference. Zhao either uses a physical roll alignment guide in the catheter to avoid roll or identifies markers or features with a known pattern with respect to the catheter in the image. However, Zhao is limited by reliance on features built into the catheter that either ensure alignment of the endoscope camera with the catheter tip or provide a known pattern of markers that needs to be visible in the image.

US 2021/0267695 to Hazelton discusses determining rotational offset of a tool with an imaging sensor with respect to a catheter. Hazelton identifies visible features, either in the catheter lumen or extending beyond the tip, that have a known orientation with respect to the catheter frame of reference. However, Hazelton also relies on visible features built into the catheter.

U.S. Pat. No. 8,147,503 to Zhao discusses tool detection in images for a surgical robot with rigid endoscope, for aspects of using the correlation of successive images to identify the tool location and for the display of markers for the tool direction of entry with respect to the FOV.

US 2023/0240513 to Shia discusses an anti-twist mechanism for a robotic endoscope camera, including a catheter tip with a ramp-like surface having an inclined flat surface formed at an angle with respect to a plane parallel to the catheter axis.

Rotation of the imaging device with respect to the tool channel and/or prevent twisting of the catheter tip with respect to the imaging device may be prevented by permanently attaching the imaging device to a housing mechanism at the tip of the continuum robot, e.g. locking the imaging device to a ramp-like surface. However, removal of the imaging device will be precluded. In contrast, systems that allow removal of the imaging device may insert/remove the imaging device through the catheter. During the insertion/removal, the imaging device may twist in an unpredictable manner.

Conventional systems and methods fail to overcome situations in which the FOV may unexpectedly change due to roll, pitch, yaw and/or walk-back of a removal imaging device relative to the continuum robot within which the imaging device is inserted, thus reducing reliability of a frame of reference between the imaging device and the tip of the continuum robot, and potentially reducing control and maneuverability of the continuum robot.

### SUMMARY

The present disclosure calibrates the view to robot control frames of reference, to overcome shortcomings of conventional systems such as those described herein.

The present disclosure relates generally to continuum robot devices and, more particularly, to devices and methods for image orientation during navigation of a continuum robot to guide interventional tools and instruments, such as inspection probes, endoscopes and/or catheters.

An aspect of the present disclosure provides an end adaptor that is configured to be inserted through a channel that extends through at least a part of a continuum robot, with the end adaptor including a first end; a second end; a body extending from the first end to the second end; and at least one protuberance extending from the body. The at least one protuberance includes a distal end, a proximal end, and a center extending between the distal end to the proximal end. The second end is configured to abut a shaft that is configured to move the end adaptor in the channel. Upon moving the end adaptor to a distal end of the channel, the first end aligns with a tip of the continuum robot, and the end adaptor precludes the first end from protruding from the distal end.

Another aspect of the present disclosure provides an inspection system that includes a continuum robot including a distal end and a proximal end, a channel, and a groove in an inner surface of the channel, the groove extending from a distal end of the channel toward the proximal end; and an end adaptor configured to travel through the channel, the end adaptor including a protuberance configured to engage the groove. Engaging the protuberance in the groove precludes independent rotation of the end adaptor.

A further aspect of the present disclosure provides a continuum robot that includes a channel and a groove provided in an inner surface of a distal end of the channel, with the groove including a lofted inner transition area at or adjacent to the distal end, and the channel being configured to accommodate an end adaptor.

Yet another aspect of the present disclosure provides an inspection method that includes moving a distal end of a continuum robot to a position at or near a region to be inspected, the continuum robot including a channel, a groove and a lofted section. The channel has a substantially circular inner surface, the lofted section being provided in a distal part of the groove. The groove is provided in the substantially circular inner surface and extending from a distal end of the channel toward a proximal end of the continuum robot, with the groove twisting in the substantially circular inner surface, as the groove extends from the distal end of the bending continuum robot. The method also includes inserting an end adaptor into a proximal end of the channel, the end adaptor including a protuberance having a width matching a width of the groove and having a profile matching a profile of the lofted section; moving the end adaptor from the proximal end to a predetermined distance from the distal end of the channel; at the predetermined distance, engaging the protuberance in the groove; between the predetermined distance and the distal end of the channel, continuing to move the end adaptor toward the distal end of the channel, with the continued movement rotating the end adaptor corresponding to the twist of the groove; engaging the protuberance in the lofted section; and performing the inspection.

These and other embodiments, objects, features, and advantages of the present disclosure will become apparent upon reading the following detailed description of exemplary embodiments of the present disclosure, when taken in conjunction with the appended drawings, and provided claims.

### BRIEF DESCRIPTION OF THE DRAWINGS

The accompanying drawings, which are incorporated herein and form part of the specification, illustrate various embodiments, objects, features, and advantages of the present disclosure.

FIG. 1A illustrates use a system according to various embodiments.

FIG. 1B illustrates components of a system according to various embodiments.

FIG. 2 illustrates a method to align an endoscopic camera view display orientation with a robot control motion direction according to an embodiment regarding continuum robot orientation display calibration for a continuum robot.

FIG. 3 illustrates a method of calibration according to an embodiment regarding continuum robot orientation display calibration for a continuum robot.

FIG. 4 illustrates modification of orientation of a displayed image according to an embodiment regarding continuum robot orientation display calibration for a continuum robot.

FIG. 5 illustrates components of a continuum robot.

FIG. 6 illustrates components for operation of a continuum robot.

FIGS. 7A and 7B are end views of a housing/camera can positioned at a distal tip of a continuum robot.

FIG. 8 is a perspective view of an end adaptor of the present disclosure.

FIG. 9 is a profile view of the end adaptor of FIG. 8 positioned at a distal end of the continuum robot.

FIG. 10 is an end view of the end adaptor of FIG. 8.

FIG. 11 is a perspective view of a continuum robot of the present disclosure with an end adaptor positioned therein.

FIG. 12 is a cut-away end view of the continuum robot and end adaptor of FIG. 11.

FIG. 13 is an end view of the continuum robot and end adaptor of FIG. 11.

FIG. 14 is a view from an imaging device within the continuum robot of the present disclosure.

FIG. 15 is a perspective view of a proximal camera connector of the present disclosure.

FIG. 16 is a perspective view of a catheter tip of the present disclosure being secured onto a continuum robot.

FIGS. 17A and 17B are profile views of a reflowed continuum robot tip and a covered reflowed continuum robot tip, respectively.

FIGS. 18A and 18B are perspective views of an end adaptor according to another embodiment the present disclosure.

FIG. 19 is a cut-away end view of the continuum robot of FIGS. 18A and 18B.

FIG. 20 is a perspective view of a tip of the present disclosure secured to a continuum robot.

FIG. 21 is a perspective view of an end adaptor according to another embodiment of the present disclosure.

FIG. 22 is an end view of the end adaptor of FIG. 21 according to an embodiment.

FIG. 23 is a perspective view illustrating a proximal lofted section of a tip according to an embodiment.

FIG. 24 is a perspective view illustrating a distal lofted section of a tip according to an embodiment.

Throughout the drawings, similar reference numerals and characters, unless otherwise stated, are used to denote like features, elements, components, or portions of the illustrated embodiments. Moreover, while the subject disclosure will now be described in detail with reference to the drawings, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION

The present disclosure generally relates continuum robots, as used for medical devices, inspection probes, related devices and method of use thereof. The embodiments of the continuum robot and portions thereof are described in terms of their state in a three-dimensional space. As used herein, the term position refers to a location of an object or a portion of an object in a three-dimensional space (e.g., three degrees of translational freedom along Cartesian X, Y, Z coordinates); the term orientation refers to the rotational placement of an object or a portion of an object (three degrees of rotational freedom—e.g., roll, pitch, and yaw); the term posture refers to the position of an object or a portion of an object in at least one degree of translational freedom and to the orientation of that object or portion of object in at least one degree of rotational freedom (up to six total degrees of freedom); the term shape refers to a set of posture, positions, and/or orientations measured along an elongated body.

As known in the field of medical devices, the terms proximal and distal are used with reference to manipulation of an end of an instrument extending from the user to a diagnostic site. In this regard, the term proximal refers to the portion (e.g., a handle) of the instrument closer to the user, and the term distal refers to the portion (tip) of the instrument further away from the user and closer to a diagnostic site, unless expressly indicated otherwise in a particular embodiment. For convenience and clarity, spatial terms such as vertical, horizontal, up, and down may be used herein. However, surgical instruments are used in many orientations and positions, and these terms are not intended to be limiting and/or absolute.

Continuum robot may generally refer to a flexible and thin tubular instrument that may be inserted through an opening, e.g. an anatomical lumen or vessel, and may perform a broad range of functions, including inspections in difficult to access locations and/or medical functions. The continuum robot includes an elongated shaft made of one or more actuatable sections. A s used herein, any of continuum robot/endoscope/catheter/probe may refer to a steerable rigid or flexible instrument.

A procedure in which an endoscope is inserted through a natural opening is called endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes. As used herein, the terms optical fiber, fiber optic, or simply fiber may refer to an elongated, flexible, light conducting conduit capable of conducting light from one end to another end due to the effect known as total internal reflection. The terms light guiding component or waveguide may also refer to, or may have the functionality of, an optical fiber. The term fiber may refer to one or more light conducting fibers.

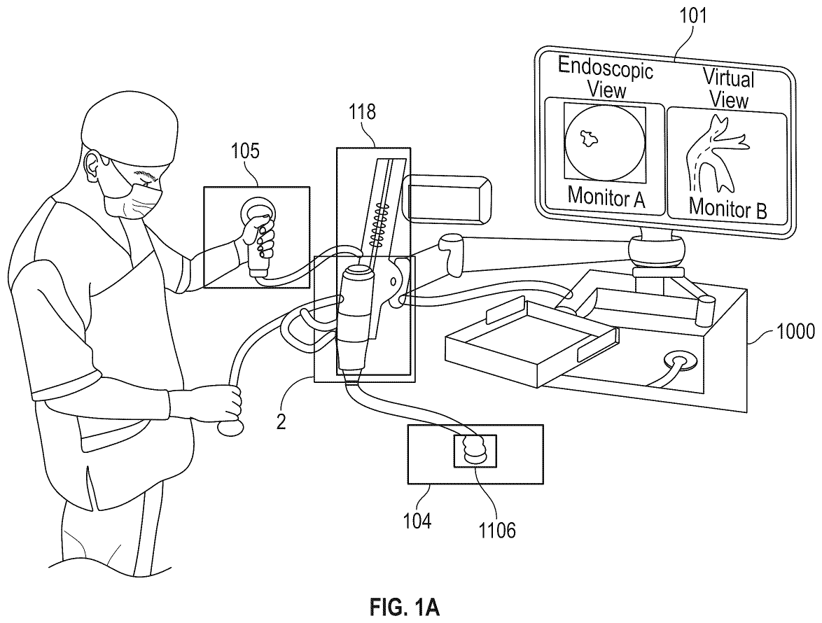

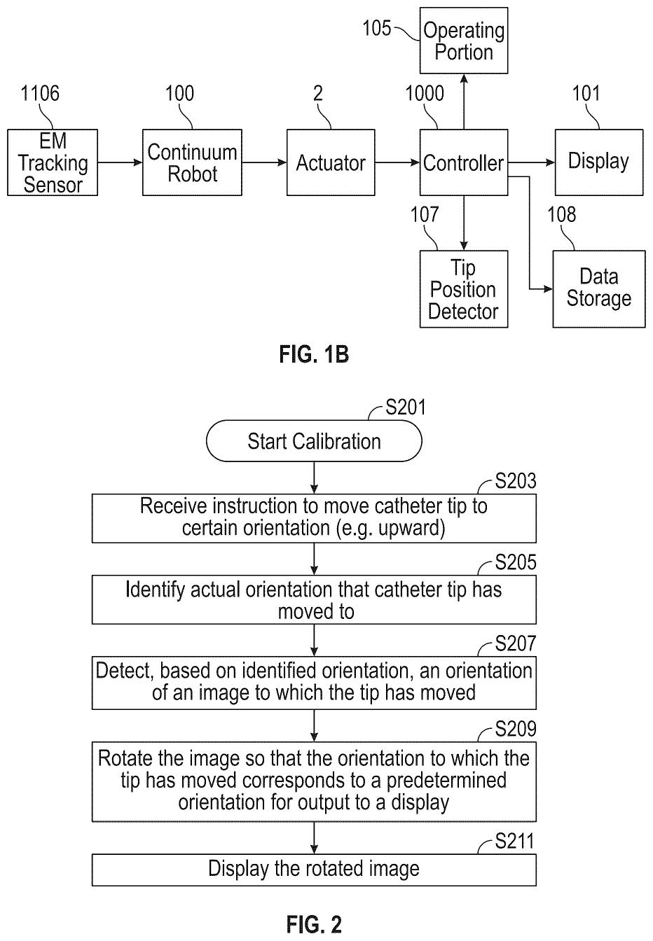

FIG. 1A illustrates use a system according to various embodiments. FIG. 1B illustrates components of a system according to the various embodiments.

As illustrated in FIGS. 1A and 1B, controller 1000 may execute one or more software programs to control displaying a navigation screen on display 101. Controller 1000 may generate a 3D model of a structure, e.g., a patient's pulmonary system, based on previously acquired images of the patient's anatomy, e.g., CT images, MRI, etc. The 3D model may be received by the system or by the controller 1000 from another device. A previously generated two-dimensional (2D) model may be used instead of a 3D model. Alternatively, the 2D or 3D model may be generated in real-time, e.g., parallel while navigating the patient's pulmonary system.

The controller 1000 may acquire position information of a continuum robot 100. The continuum robot 100 may include an electro-magnetic (EM) tracking sensor 1106 on a distal end thereof to track position and orientation thereof. The controller 1000 may acquire position information directly from a tip position detector 107, via the EM tracking sensor 1106.

The controller 1000 may receive information regarding a location of a tip of the continuum robot 100, i.e., positional information, from the tip position detector 107. The positional information may be stored in data storage 108, i.e., a memory, which may store images of a segmented model created from a preoperative CT scan. The controller 1000 may include at least one read only memory (ROM), at least one central processing unit (CPU), at least one random access memory (RAM), at least one input and output (I/O) interface, and at least one hard disc drive (HDD). A solid-state drive (SSD) may be used with or instead of the HDD. The at least one ROM includes a non-transitory computer readable storage medium that stores instructions that, when executed, cause processor(s) of the controller 1000 to conduct methods, processes, and steps described herein.

The ROM and/or HDD may be used to store software programs, and the RAM may be used as a working memory, and the ROM, the HDD and/or the RAM may store computer-executable code for operating the system according to an embodiment.

The CPU may be used to execute one or more software programs and/or code stored in the ROM, the HDD and/or the RAM. The I/O interface may receive positional information for input to the controller 1000 and may output information for displaying the navigation screen to the one or more displays. The navigation screen may be generated by the software program but may be generated by firmware.

The controller 1000 may send input signals to control an actuator 2 and/or a rail based on user manipulation via one or more operation portions 105, e.g. handheld controller and/or joystick. The input signals are recorded and stored in data storage 108. The continuum robot 100, which may be disposable, may be attachable/detachable to/from the actuator 2. The actuator 2 may proceed/retreat along the rail 118, to advance/withdraw the continuum robot 100 into/from a patient's body or structure that is to be inspected.

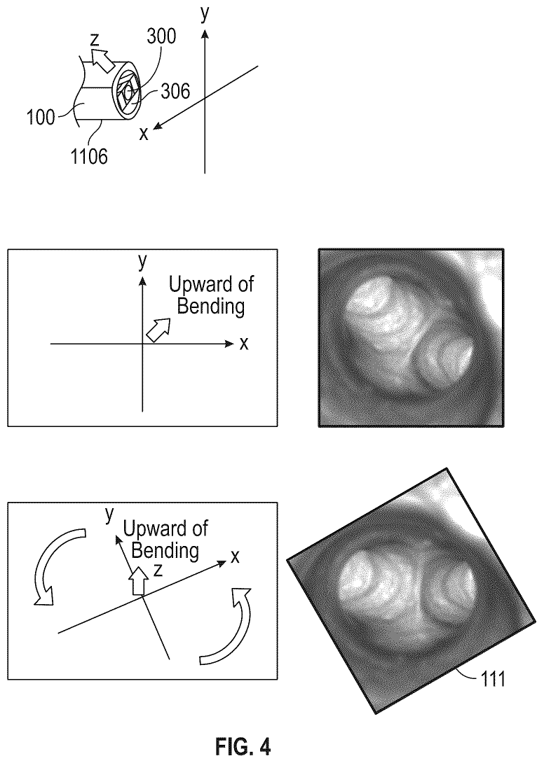

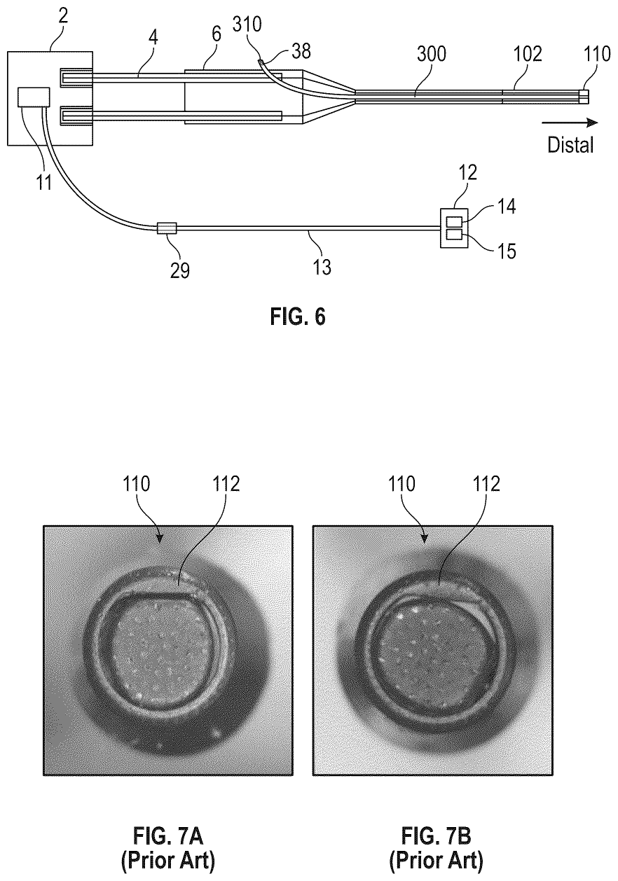

The continuum robot 100 may include one or more sensors 306 that may include one or more of a tracking sensor, camera, image capture device, accelerometer, gyroscope, force detector, temperature detector, and/or position sensor. The continuum robot 100 may include a channel 300 (FIGS. 4, 6) for insertion of an imaging capture device, camera and/or other sensor 306 therethrough. The channel 300 may be configured to also receive medical tools, e.g., an EM tracking sensor 1106, a laser, forceps, a needle or other biopsy tools, and/or a basket catheter, etc., without limitation thereto. The EM tracking sensor 1106 may be attached to the tip of the continuum robot 100. In certain embodiments, the continuum robot 100 may include the EM tracking sensor 1106 on a distal end thereof. An image capturing tool, e.g., an imaging device, may be inserted through the channel 300 of the continuum robot 100.

The continuum robot 100 may include a proximal section, a middle section, and a distal section, bendable by operation of a plurality of driving wires, i.e., driving liner members, with the posture of the continuum robot 100 being supported by supporting wires, i.e., supporting liner members. The plurality of driving wires are connected to the actuator 2, which includes one or more motors configured to drive sections of the continuum robot 100 by pushing and/or pulling the driving wires. A mechanical frame of reference is obtained by at least one of visual inspection or feedback from the plurality of driving wires when operated to bend the continuum robot 100.

FIG. 2 illustrates a method to align an endoscopic camera view display orientation with a robot control motion direction according to an embodiment regarding calibration of an imaging device of a continuum robot.

As shown in FIG. 2, a method of calibration starts at step S201 and, at step S203, the controller 1000 receives instruction to move the tip, i.e., the distal end of continuum robot 100, to a predetermined orientation, e.g., upward, toward a target, by a predetermined amount toward a new position. First data corresponding to a first position of a tip of the distal end may be received from the EM tracking sensor 1106 and the first data may be stored in data storage 108.

The data storage 108 may also store a plurality of images of an anatomical model obtained by pre-operative and/or intra-operative imaging. For each predetermined pathway section of the anatomy, each image of the plurality of images of the anatomical model includes a preferred orientation.

A command to move the tip to a predetermined amount to a predetermined orientation is received and controller 1000 controls at least one actuator of the plurality of actuators to move the tip based on the received command. Second data corresponding to a second position of the tip may be received from the EM tracking sensor 1106 and an image corresponding to the second position of the tip may be received from an imaging device of the sensor 306. The received second data and image may be stored in data storage 108 and an image of a stored plurality of images corresponding to a predetermined orientation at the second position may be obtained from the data storage 108.

Calibration may be performed upon receipt of each instruction to move the tip by a predetermined orientation. Alternatively, calibration may start on either user request or change of a steering mode of the bendable body, e.g., change from a drive mode to a targeting mode. Upon change to the steering mode, a prompt may be output for a user to input a command to move the tip by a predetermined amount to change at least one of a position or an orientation. Alternatively, upon change to the steering mode, an instruction may be automatically output to move the tip to the at least one predetermined orientation or position.

At step S205 identification is made of at least one of an actual position to which the tip has moved in accordance with the received instruction. As described herein, positions of the tip before and after movement of the continuum robot 100 may be stored in data storage 108. Based on at least one of comparison of the stored tip positions and comparison of the received image with the obtained image, a determination may be made of an orientation of the tip in the position to which the tip has moved.

At step S207 a determination may be made, based on comparison of the determined orientation of the tip in the position to which the tip has moved with previously acquired images of the patient's anatomy, of a change amount to modify display of an image of the target.

At step S209 an orientation of the received image may be modified to correspond to an orientation of the obtained image. Thus, the output image may be rotated so that the orientation to which the tip moves corresponds to a predetermined orientation on a display and, at step S111, the rotated image is displayed. Thus, a surgical apparatus may be provided that includes a continuum robot with a body having a proximal portion and an articulated distal portion configured to house at least a sensor and an imaging device, with the sensor being configured to detect a position of the distal portion; a plurality of actuators operatively coupled with the articulated distal portion; a memory configured to store a plurality of images of an anatomical model, wherein, for each predetermined pathway section, e.g., pathway in the anatomy, each image of the plurality of images includes a preferred orientation; and a controller in operational communication with the sensor, the imaging device, the plurality of actuators, and the memory. The controller may be configured to receive, from the sensor, first data corresponding to a first position of a tip of the distal portion; store, in the memory, the first data; receive a command to move the tip a predetermined amount to a predetermined position; control at least one actuator of the plurality of actuators to move the tip based on the received command; receive, from the sensor, second data corresponding to a second position of the tip; receive, from the imaging device, an image corresponding to the second position; store, in the memory, the received second data and image; obtain, from the memory, an image and corresponding predetermined orientation from among the stored plurality of images corresponding to the second position; compare the received image with the obtained image; and modify an orientation of the received image to correspond to the corresponding predetermined orientation of the obtained image.

The imaging device provided on the sensor 306 may to twist relative to the body of the continuum robot 100 and the sensor 306 may be configured to measure movement in three or more degrees of freedom of the articulated distal portion of the continuum robot 100. A two-dimensional display may be provided that is configured to output the received image with the modified orientation.

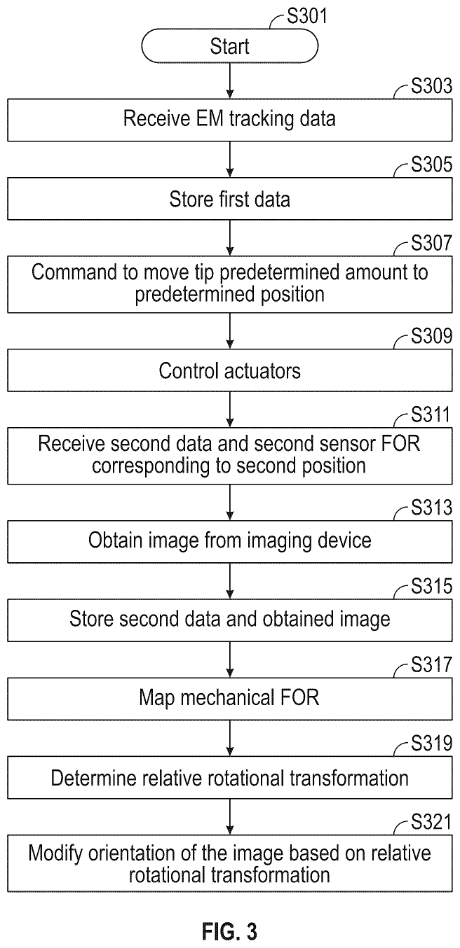

FIG. 3 illustrates a method of calibration according to an embodiment regarding catheter orientation display calibration for a continuum robot.

A method of calibration may start at step S301. At step S303, the controller 1000 may receive from the EM tracking sensor 1106 at least one of first data and a first sensor frame of reference that correspond to a first position (L1) of a tip of the distal portion of the continuum robot 100. The first data may include first vector coordinates [x₁, y₁, z₁]. At step S305 the first data may be stored in data storage 108. At step S307 a command may be received to move the tip a predetermined amount to a predetermined position. At step S309 the controller 1000 may control at least one actuator of the plurality of actuators to move the tip based on the received command. At step S311 second data and a second sensor frame of reference corresponding to a second position of the tip may be received from the EM tracking sensor 1106. The second data may include second vector coordinates [x₂, y₂, y₂] corresponding to a second position. At step S313 an image may be obtained from the imaging device of the sensor 306 and, at step S315, the second data and the image may be stored in data storage 108. At step S317 a mechanical frame of reference based on the second vector coordinates [x₂, y₂, y₂] is mapped and, at step S319, a relative rotational transformation between the first sensor frame of reference and second sensor frame of reference may be determined based on a detected movement vector. That is, a twist of the continuum robot 100 results in an offset in the design orientation, and a twisted frame of reference is identified in S317. At step S321 an orientation of the image based on the relative rotational transformation may be modified.

At least one indicator of the relative rotational transformation may be displayed and orienting the image based on the relative rotational transformation aligns a camera view display orientation with a control motion direction of the continuum robot.

In response to receiving another command to move the tip another predetermined amount to another predetermined position, another orientation of the image may be performed based on another relative rotational transformation. Another relative rotational transformation may be determined based on change of pose of the continuum robot and another relative rotational transformation may be determined based on a change between control modes.

FIG. 4 illustrates modification of orientation of a displayed image according to an embodiment regarding catheter orientation display calibration for a continuum robot. In FIG. 4, an output of a calibration process for aligning the orientation of the continuum robot camera FOV that is displayed with the robot control motion direction. Subsequent adjustments may be made to account for rotation of the camera relative to the continuum robot 100 during navigation, until the moment that the camera is removed. The display orientation adjustments may be reflected in the virtual view, to be used as reference for further continuum robot 100 motion.

The calibration may be continuously performed during navigation, every time there is a change in posture of the continuum robot 100 and corresponding change of image information. Alternatively or in addition, the calibration may be an explicit step, either requested by the user or by the system upon relevant state changes, such as when switching robot control modes. The user may then be requested to initiate motion of the continuum robot 100, or the system may automatically perform a predefined motion to update the posture.

Thus, the calibration rotates the image so that the display orientation matches one of a control direction of motion and a predetermined orientation. Alternatively, markers, e.g., arrows, may be displayed on the image display to visualize the direction of motion orientation, such as a compass style display. As an additional alternative, mapping of the robot control input device, e.g., joystick, may be modified so that the control direction corresponds to the image frame of reference.

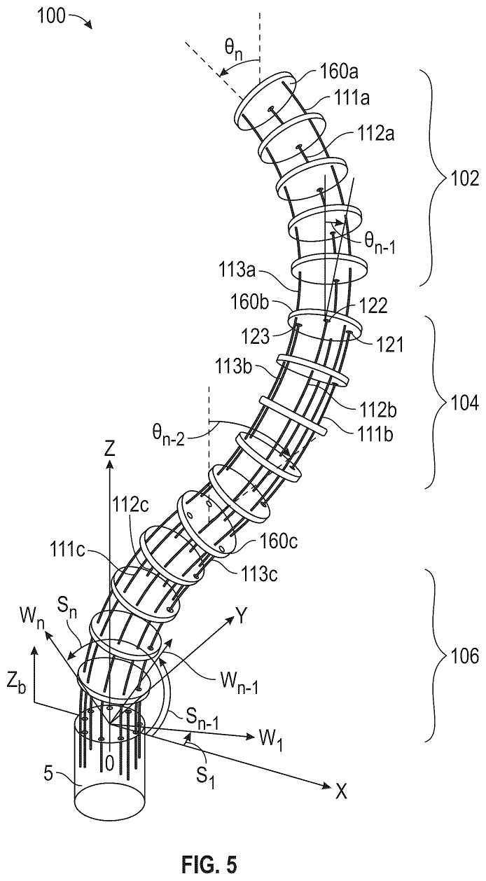

FIG. 5 illustrates components of a continuum robot.

As shown in FIG. 5, the continuum robot 100 includes push/pull drive wires 111b, 112b and 113b, which are connected to connection portions 121, 122 and 123, respectively, found on an end disc 160b, for controlling the middle bending section 104. Additional drive wires (three for each of the bendable sections 102 and 106) 111a, 111c, 112a, 112c, 113a, 113c, are attached at the distal ends of each bendable section 102 and 106, to the respective end disc 160a and 160c. Each bending section is similarly operated. Thus, the description of one bending section, i.e., the middle bending section 104, will be recognized to apply to the other sections. Posture of the bending section 104 is controlled by pushing and pulling the wires 111b to 113b by using actuators, with l_{d}=the length of the central axis a bending section; θₙ=the bending angle of the distal end; ζₙ=the rotational angle of the distal end; ρₙ=the radius of curvature of a bending section. Drive wires 111a . . . c, 112a . . . c, 113a . . . c are also referred to as at least one drive wire 4 herein.

FIG. 6 illustrates components for operation of a continuum robot.

The continuum robot 100 includes a catheter shaft, a/k/a bendable part, that may be disposed on a base stage to move in a longitudinal direction, to advance, retard and/or retract into a target structure. The longitudinal direction aligns with a longitudinal axis, a/k/a roll axis, running through the body of the continuum robot from hub to distal end.

As illustrated in FIG. 6, a system for operating the continuum robot may include an actuator 2 (e.g., driving unit or driver) for applying a driving force on the drive wires (tendons or driver(s)) of the continuum robot 100 via an operation console (controller/control system), which may include push-button, thumb-stick, and/or joystick, and/or navigation software.

An operation console may indicate an amount of driving force to the base stage and, independently, to the actuator 2. The operation console may include dedicated hardware including a field-programmable gate array (FPGA) and the like; and/or may be a computer including a storage unit, a work memory, and a CPU. Where the operation console is a computer, the storage unit may be a memory that stores a software program corresponding to a control system algorithm and the CPU may expand the program in the work memory, and may execute the program line by line, for the computer to function as the operation console. In either case, the operation console may communicably connect with the base stage and the actuator 2, and the operation console may send signals representing the driving amount and configuration to these control targets, which may be imputed by an end user through push buttons, joystick or the like. Thus, the continuum robot 100 includes at least one distal bending section, with robotic control for insertion and removal of the continuum robot 100 from the target for operation during lung biopsies, medical procedures, and similar operations.

Posture and/or pose of the catheter shaft may be controlled by application of push/pull force(s) on at least one drive wire 4. A catheter shaft, a/k/a bendable part, includes at least one distal bending section 102, with at least three drive wires terminating in each of the at least one distal bending section 102 to control bend angle and plane. The actuator 2 may selectively push/pull the at least one drive wire 4 to control the bend angle and/or plane of the distal bending section 102. The at least one drive wire 4 may be spaced apart from another at least one drive wire 4 at a pitch diameter of 22 mm.

In an embodiment, misalignment of the image and tip frames of reference is accounted for, without having to rely on visible features built into or embedded in the continuum robot 100. Thus, a surgical apparatus may be provided that includes a continuum robot that includes a body having a proximal portion and an articulated distal portion configured to house at least a sensor and an imaging device, wherein the sensor is configured to detect a position of the distal portion; a plurality of actuators operatively coupled with the articulated distal portion; a memory configured to store a plurality of images of an anatomical model, wherein, for each predetermined pathway section, each image of the plurality of images includes a preferred orientation; and a controller in operational communication with the sensor, the imaging device, the plurality of actuators, and the memory.

In an embodiment, the controller may be provided that is configured to receive, from the sensor, first data corresponding to a first position of a tip of the distal portion; store, in the memory, the first data; receive a command to move the tip a predetermined amount to a predetermined position; control at least one actuator of the plurality of actuators to move the tip based on the received command; receive, from the sensor, second data corresponding to a second position of the tip; receive, from the imaging device, an image corresponding to the second position; store, in the memory, the received second data and image; obtain, from the memory, an image and a corresponding predetermined orientation from among the stored plurality of images corresponding to the second position; compare the received image with the obtained image; and modify an orientation of the received image to correspond to the corresponding predetermined orientation of the obtained image.

In an embodiment, the imaging device may be configured to twist relative to the body. Also, a two-dimensional display may be configured to output the received image with the modified orientation. The sensor may be configured to measure movement in three or more degrees of freedom of the articulated distal portion of the body. The proximal portion may include a passively flexible unarticulated portion and the articulated distal portion may be configured for insertion into a patient through an aperture.

In an embodiment, a surgical apparatus may be provided that includes a continuum robot that includes a body having a proximal portion and an articulated distal portion configured to house at least a sensor and an imaging device, wherein the sensor is configured to detect a position of the distal portion; a plurality of actuators operatively coupled with the articulated distal portion; a memory; and a controller in operational communication with the sensor, the imaging device, the plurality of actuators, and the memory. The controller may be configured to receive, from the sensor, first data and a first sensor frame of reference corresponding a first position of a tip of the distal portion, wherein the first data includes first vector coordinates; store, in the memory, the first data; receive a command to move the tip a predetermined amount to a predetermined position; control at least one actuator of the plurality of actuators to move the tip based on the received command; receive, from the sensor, second data and a second sensor frame of reference corresponding to a second position of the tip including second vector coordinates, wherein the second data includes second vector coordinates; obtain, from the imaging device, an image; store, in the memory, the second data and the image; map a mechanical frame of reference based on the second vector coordinates; detect a movement vector, based on the first vector coordinates and the second vector coordinates; determine a relative rotational transformation between the first sensor frame of reference and second sensor frame of reference, based on the detected movement vector; and modify an orientation of the image based on the relative rotational transformation.

In an embodiment, the controller may be configured to display at least one indicator of the relative rotational transformation and orienting the image based on the relative rotational transformation may align a camera view display orientation with a control motion direction of the continuum robot. In response to receiving another command to move the tip another predetermined amount to another predetermined position, the controller may perform another orientation of the image based on another relative rotational transformation. The controller may determine another relative rotational transformation based on change of pose of the continuum robot. The controller is configured to determine another relative rotational transformation based on a change between control modes.

The disclosure set forth below of the end adaptor, the inspection system, the continuum robot with lofted inner transition area, and methods of use of same allows a user to insert/remove the imaging device through the continuum robot to a tip lock position within a narrow range of lateral and radial positioning. The disclosure set forth below also ensures a clear FOV without the imaging device protruding beyond a tip of the catheter/probe and avoiding unexpected changes of orientation of the FOV. In addition, the disclosure that is set forth below provides guidance for expedited alignment of the imaging device at the tip.

Also illustrated in FIG. 6 are a hub 6 provided at a proximal end of the continuum robot 100, a tip 110 provided at the distal end of the continuum robot, the channel 300 that traverses from at or adjacent to the proximal end to the distal end, and a channel access port 310, a/k/a tool insertion slot. The drive wires extend through the hub 6 in a transverse direction T_{D} with proximal ends of the drive wires 4 exposed to be releasably attached to respective tractors of the actuator 2. The actuator 2 may include a channel access port 310. The housing/camera can 12 may enter the continuum robot 100 via the channel access port 310. The housing/camera can 12 may include a light source, e.g. a light emitting diode (LED) 15 and an image sensor 14, e.g. a low power consumption complementary metal-oxide-semiconductor (CM OS) image sensor 14, provided on a shaft 13, e.g. camera shaft extrusion, with a proximal camera connector 29. A camera port 11 may be provided on the actuator from which the housing/camera can 12 may extend, for a user to insert into the channel access port.

A length of the shaft 13 between the proximal camera connector 29 and the end adaptor 500/housing/camera can 12 generally matches a length of the channel 300 from the access port 310 to the tip 110. When the end adaptor 500/housing/camera can 12 is fully inserted into channel 300, the proximal camera connector 29 will generally be adjacent to the access port 310.

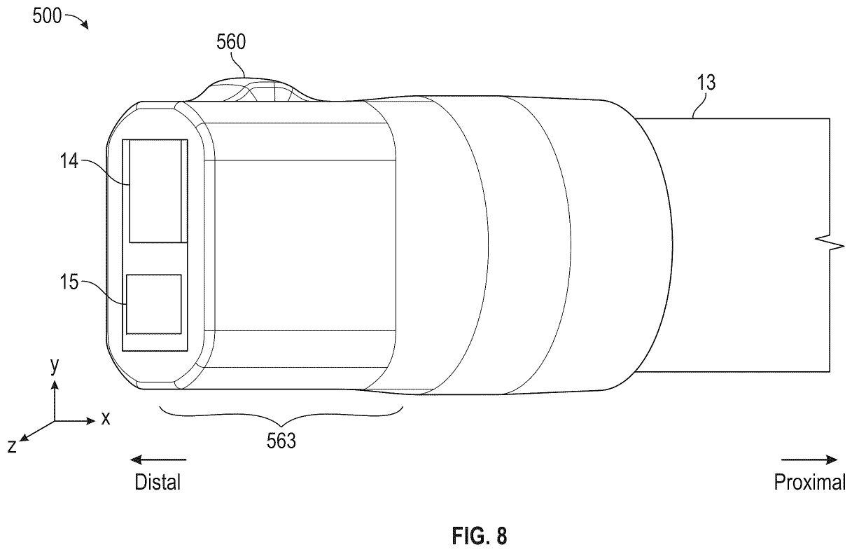

As illustrated in FIG. 8, a protuberance 560 may be provided at a predetermined radial position on an end adaptor 500, within which the housing/camera can 12 may be provided. Full insertion of the end adaptor 500/housing/camera can 12 into channel 300 will longitudinally move the protuberance 560 to a groove 320 that is provided in an inner surface of the channel 300.

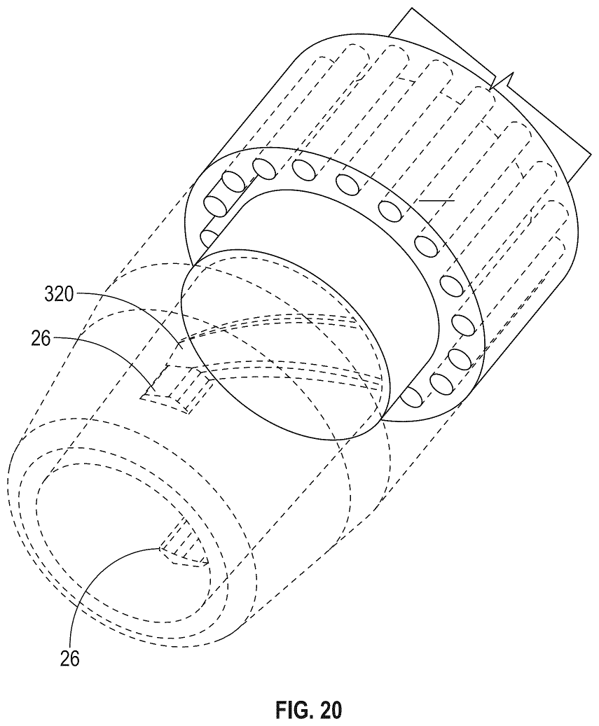

As illustrated in FIG. 6, an alignment indicator 38, i.e., alignment marking, may be provided at a predetermined radial position at or adjacent to a proximal end of the channel 300. The alignment indicator 38 is visible to a user of the hub 6 and/or inspection system. The predetermined radial position of the alignment indicator 38 corresponds to the radial position of the groove 320 (FIG. 20).

As illustrated in FIG. 15, the proximal camera connector 29 may include a fin 39 at a predetermined radial position. The fin 39 functions as a proximal aligner. That is, when a user inserts the end adaptor 500/housing/camera can 12 into the channel access port 310, the user aligns the fin 39 with the alignment indicator 38 that is provided on the channel access port 310. Such alignment radially aligns the protuberance 560 with the groove 320.

FIGS. 7A and 7B are end views of a housing/camera can positioned at a distal tip of a continuum robot.

As illustrated in FIG. 7A and FIG. 7B, the distal end 110 of the continuum robot includes a flat portion 112 that aligns with a corresponding flat portion of a housing/camera can 12, when the housing/camera can 12 is inserted through the channel 310 to predetermined radial and longitudinal positions, to reduce rotation of the housing/camera can 12. In FIG. 7A, the housing/camera can 12 is advanced to the radial and longitudinal positions at the distal end of the continuum robot to the tip 110, thereby aligning the corresponding flat portions. In FIG. 7B, the housing/camera can 12 is not advanced to the longitudinal position at the distal end of the continuum robot. Thus, the housing/camera can 12 rotates an unspecified amount and the corresponding flat portions do not align, resulting in a FOV that is not properly oriented.

### <End Adaptor>

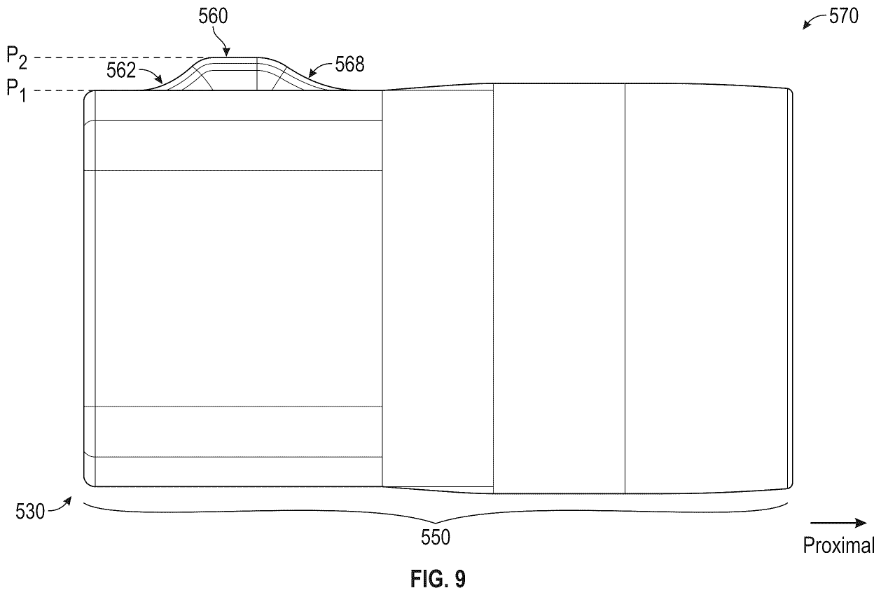

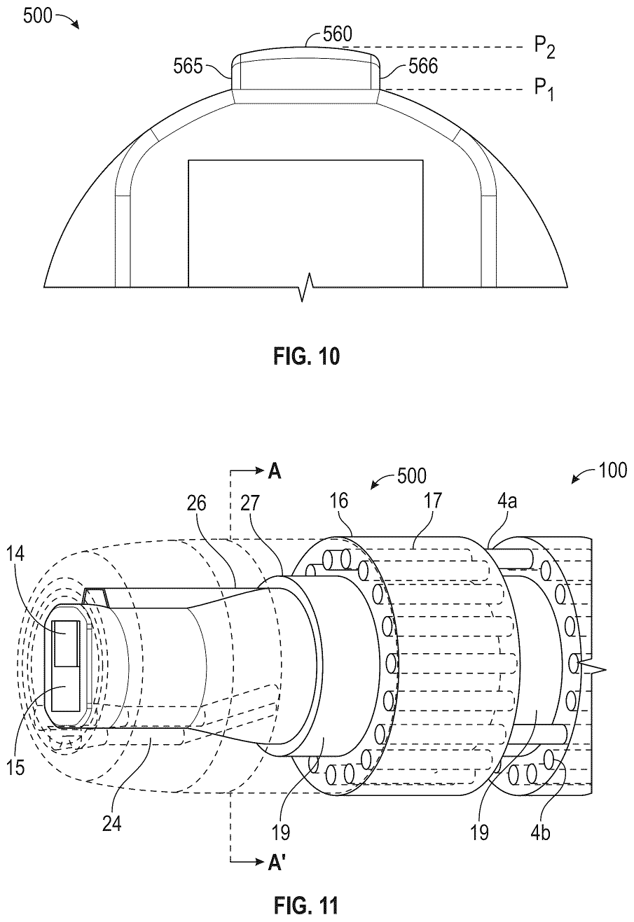

FIG. 8 is a perspective view of an end adaptor of the present disclosure. FIG. 9 is a profile view of the end adaptor of FIG. 8. FIG. 10 is an end view of the end adaptor of FIG. 8. The end adaptor 500 illustrated in FIGS. 8-10 may be inserted through the channel 300 extending through at least a part of a bendable section of the continuum robot 100, as described with reference to FIGS. 1-6.

The end adaptor 500 may include a first end 530, a second end 570, a body 550 extending from the first end 530 to the second end 570, and at least one protuberance 560 that extends from the body 550 in a transverse direction T_{D} (the Y direction in FIG. 8) that is substantially parallel to a longitudinal direction L_{D} of the bendable section. The at least one protuberance 560 includes a distal end 562, a proximal end 568, and a center 563 therebetween. As illustrated in FIGS. 89-10, opposite edges of the center may be rounded in the transverse direction T_{D}.

The first end 530 may have a substantially rectangular shape and the second end 570 may have substantially circular shape. The second end 570 may abut, i.e., connect to/receive, the shaft 13. As illustrated in FIGS. 8 and 9, the first end may include rounded corners between each side of the substantially rectangular shape.

The shaft 13 may be utilized to move the end adaptor 500 along in the channel 300 in the longitudinal direction L_{D}. When the end adaptor 500 is moved into alignment with a distal end of the channel 300, the first end 530 aligns with the tip 110 of the continuum robot 100.

When aligned with the tip 110 of the continuum robot 100, the first end 530 does not protrude from the distal end. The center includes a substantially flat portion 564 that corresponds to a predetermined position of an imaging device to be secured by the end adaptor 500. The predetermined position of the imaging device may align with a maximum outside diameter of the imaging device. Aligning the first end 530 with the tip 110 of the continuum robot 100 provides a predefined FOV for a user to perform imaging by an imaging device that is integral with or secured by the end adaptor 500.

As illustrated in FIG. 13, the end adaptor 500 may include a light source 15 and an image sensor 14 at the first end 530 thereof, for performing optical and other imaging types, for use in medical, manufacturing, inspection and related fields.

FIGS. 9 and 10 illustrate a first plane P1 and a substantially parallel second plane P2. The first plane P1 is a maximum extension of the protuberance 560 above the body of the end adaptor 500 at the second plane P2. The first plane P1 extends along an exterior of the body 550 at a lower end of the at least one protuberance 560. The second plane P2 extends along an apex of the substantially flat portion 564.

As illustrated in FIG. 10, a first wall 565 may be provided that extends along a side of the at least one protuberance 560 and a second wall 566 may be provided extending along an opposite side of the at least one protuberance 560. The first wall 565 and the second wall 566 may be substantially parallel and extend from a distal end 562 to a proximal end 568 in a longitudinal direction L_{D} (X direction). Upper parts of the first wall 565 and the second wall 566 connect with a respective bottom portions of the respective rounded edges of the center, the first wall 565 and the second wall 566 set a width of the at least one protuberance 560, and the width of the at least one protuberance 560 is configured to fit within the channel 300.

As also illustrated in FIGS. 9 and 10, the distal end 562 and the proximal end 568 of the protuberance 560 of the end adaptor 500 may each have substantially concave regions between the first plane P1 and the second plane P2. Thus, the at least one protuberance 560 is configured to engage a groove 320 in an inner surface of the channel 300 (FIGS. 11-13, 20). At least this engaging precludes independent rotation of the end adaptor 500 independent of the continuum robot 100.

The groove 320 may extend from a position adjacent to a distal end of at least one bending section of the continuum robot.

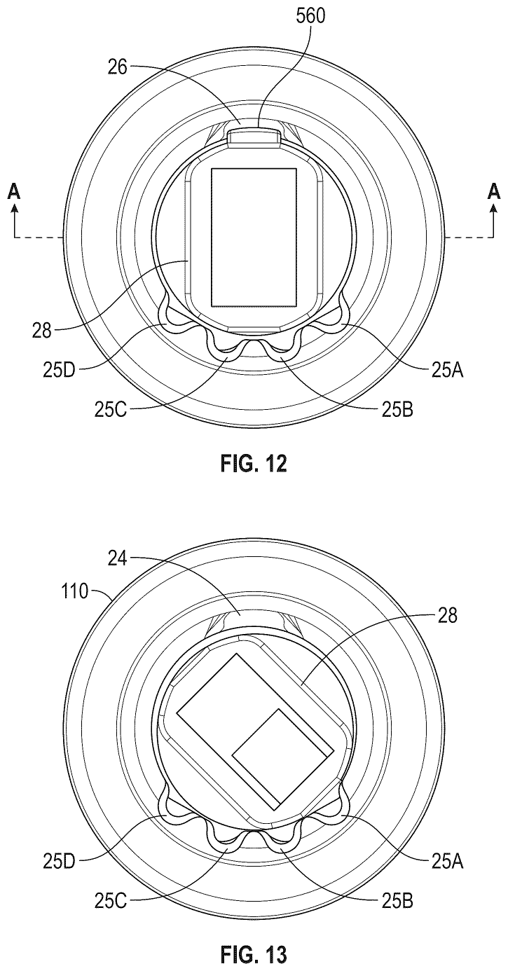

FIG. 11 provides a perspective view of a continuum robot of the present disclosure with the end adaptor 500 positioned therein. FIG. 12 is a cut-away end view of the continuum robot and continuum robot of FIG. 11. FIG. 13 is an end view of the continuum robot and continuum robot of FIG. 11.

FIG. 11 illustrates, on a right side thereof, a distal end of the continuum robot 100 with a drive ring 16, a guide ring 17, and guide wires 4a, 4b. A catheter tip proximal bore 27 with inner cover 19 extending from the distal end of the continuum robot 100 into the end adaptor 500 is also illustrated. A lofted inner transition area 26, i.e., lofted area or lofted section, and tip cutouts 24 are also illustrated. Engaging the at least one protuberance 560 with the lofted inner transition area 26 precludes longitudinal walk-back of the inserted image sensor 14 that may occur result from flush operation pressure applied against the end adaptor 500.

FIG. 12 shows a camera distal profile 28 inserted within the channel 300, with the protuberance 560 aligned with and inserted into the tip cutout 24. On an opposite side of the protuberance 560, a plurality of tip grooves 25a, 25b, 25c are provided. The camera tip grooves 25a, 25b, 25c allow for flow from the tip 110 into the channel 300 while the end adaptor 500/housing/camera can 12 remains inserted into channel 300. Such flow may be used for flush and other operations performed by the continuum robot.

FIG. 13 shows the camera distal profile 28 inserted in the channel 300, with the protuberance 560 not aligned with and not inserted into the tip cutout 24. In FIG. 13, the protuberance 560 is partially inserted into a catheter tip groove 25a.

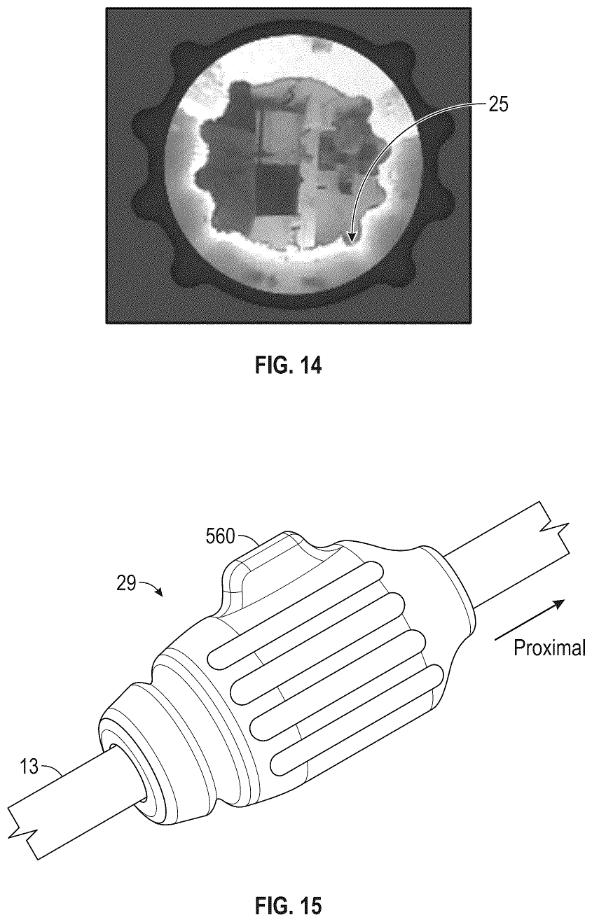

FIG. 14 is a view from an imaging device within the continuum robot of the present disclosure. As illustrated, a plurality of equidistantly spaced apart and/or symmetrically arranged grooves are provided. The symmetrical arrangement of the plurality of grooves may provide a flower-shaped opening at the distal end to the continuum robot 100.

FIG. 15 is a perspective view of a proximal camera connector of the present disclosure.

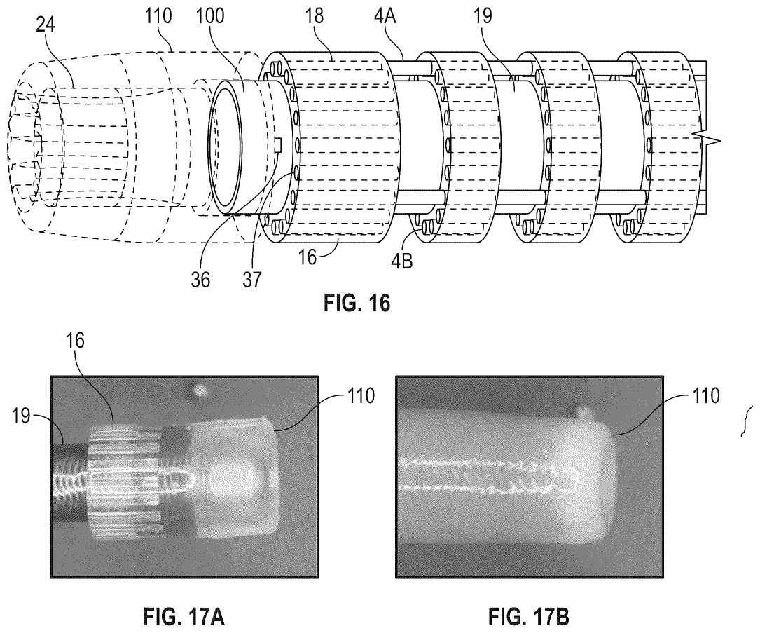

FIG. 16 is a perspective view of a tip 110 of the present disclosure being secured onto the continuum robot 100. As illustrated on a right side of FIG. 16 and as described herein, a distal end of the continuum robot 100 may include a drive ring 16, guide wires 4a, 4b, and an anchor 18 for guide wire 4a. FIG. 16 illustrates the tip 110 partially removed from the continuum robot 100, with tip cutouts 24 longitudinally provided along at least one inner surface of the tip 110, with a fastener 36 extending toward a receiver 37 for locking therein.

### <Continuum Robot>

FIGS. 17A and 17B are profile views of a reflowed tip 110 of the continuum robot 100 and a covered reflowed continuum robot tip 110, respectively. Regarding reflow, an outer cover of the continuum robot 100 may be formed of a thermoplastic material and is configured to be thermally bonded to a tip of the continuum robot 100.

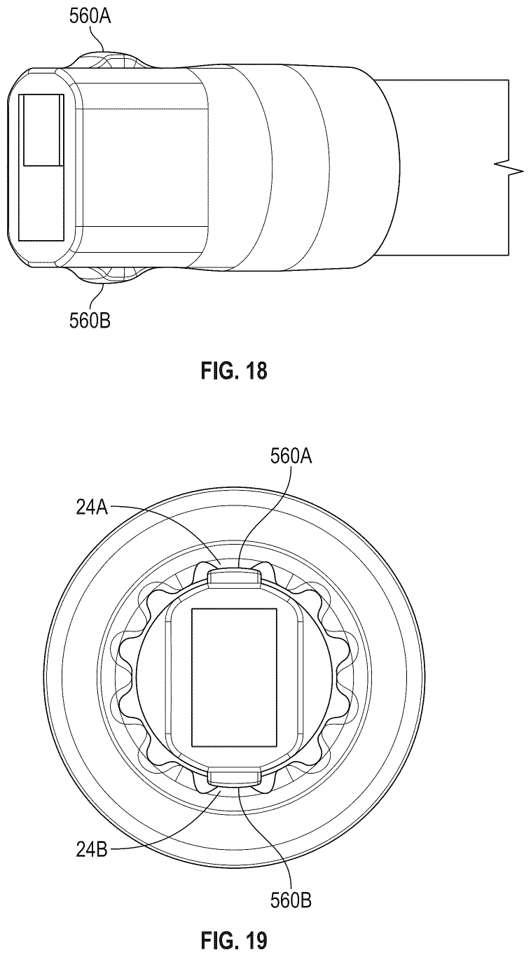

FIGS. 18A and 18B are perspective views of an end adaptor according to another embodiment the present disclosure. FIG. 18A is a perspective view of the end adaptor partially reflowed. FIG. 18B is a perspective view of the end adaptor fully reflowed.

FIG. 19 is a cut-away end view of the continuum robot of FIGS. 18A and 18B. FIG. 20 is a perspective view of a tip of the present disclosure secured to a continuum robot.

In contrast to the flat portion of the tip 112 illustrated in FIGS. 7A and 7B, in the embodiment of FIG. 20, at least one lofted inner transition area 26 is provided in the channel 300 at a distal end of groove 320. The at least one lofted inner transition area 26 is arranged to abut the substantially flat portion 564 (FIG. 9) when the end adaptor 500 is fully inserted in the channel 300. The substantially flat portion 564 may have a length corresponding to a length of the corresponding flat portion of the lofted inner transition area 26. The flat portion 340 may form part of a lofted inner transition area 26. For clarity, FIG. 20 does not illustrate the tip grooves that may be provided therein, as illustrated in FIG. 12.

Positioning the substantially flat portion 564 in the corresponding flat portion of the lofted inner transition area 26 precludes longitudinal and rotational movement of the end adaptor 500 relative to of the continuum robot 100. The preclusion of longitudinal movement suppresses walk-back of the inserted image sensor 14 that may occur due to pressure against the end adaptor 500 when a flush operation is performed. Precluding both longitudinal and rotational movement substantially eliminates pitch and/or yaw of the end adaptor 500 relative to the continuum robot 100.

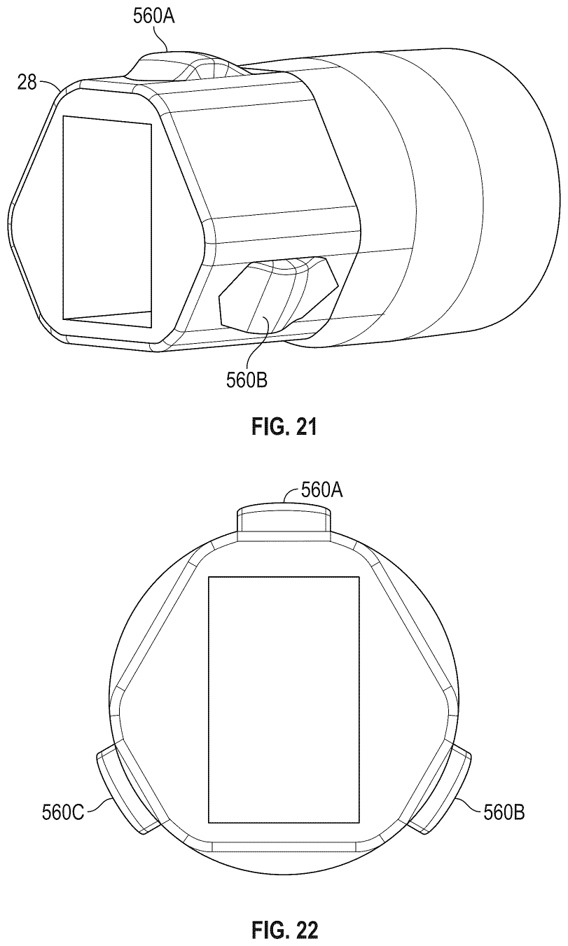

FIG. 21 is a perspective view of an end adaptor according to another embodiment of the present disclosure. FIG. 22 is an end view of the end adaptor of FIG. 21.

As illustrated, the end adaptor 500 may be provided with one or more than one protuberance 560A, 56B, 560C, and the channel 300 of a system for use therein may include a corresponding number of grooves. As illustrated in FIGS. 19 and 20, the end adaptor 500 may be provided with a first protuberance 560A and a second protuberance 560B, which may be positioned opposite to the first protuberance 560A. The first protuberance 560A and the second protuberance 560B may be symmetrically arranged, for respective alignment with a first groove and a second groove and first and second lofted inner transitions that are equidistantly and/or symmetrically arranged.

Accordingly, a continuum robot 100 is provided that includes at least one bending section having an inner working channel 300 that extends therethrough in a longitudinal direction L_{D}. The continuum robot 100 includes a first groove 320 provided in an inner surface of the channel 300 at a distal end of the at least one bending section. The first groove 320 may include a lofted inner transition area 26 at or adjacent to the distal end. The channel 300 may be configured to accommodate an end adaptor 500. The end adaptor 500 may include a protuberance 560 having a profile matching a profile of the lofted inner transition area 26. Moving the end adaptor 500 within the channel 300 to the distal end engages the protuberance 560 in the lofted inner transition area 26. Engaging the protuberance 560 in the lofted inner transition area 26 fixes a position of the end adaptor 500.

The fixed position aligns a first end 530 of the end adaptor 500 with a tip 110 of the continuum robot 100 and provides a predefined field of view for an imaging device integral with or secured by the end adaptor 500, without the first end 530 protruding beyond the distal end.

A distal end of the end adaptor 500 may be substantially rectangular shaped. When the end adaptor 500 is inserted into the tip, a clearance is formed between the channel 300 and the substantially rectangular shaped housing of the end adaptor 500 to allow fluid flow through the channel 300 without removing the end adaptor 500 from the tip.

The end adaptor 500 may include a protuberance 560 having a profile matching a profile of the lofted inner transition area 26. Engaging the protuberance 560 in at least one of the lofted inner transition area 26 and the channel 300 maintains a predetermined orientation between the end adaptor 500 and the at least one bending section. The predetermined orientation aligns the at least one bending section with an imaging device integral with or secured by the end adaptor 500.

The protuberance may include a biasing member, e.g. a spring or plastic part, that applies a force to extend the protuberance to a maximum height thereof.

The continuum robot may at least one of change of pose and/or posture of the at least one bending section, with the end adaptor 500 including a protuberance 560 and the hub including at least one alignment indicator 38 configured to indicate a position of at least one of the imaging device and/or the protuberance 560 relative to the channel 300. Thus, proper alignment by a user of the camera/protuberance in the channel is simplified.

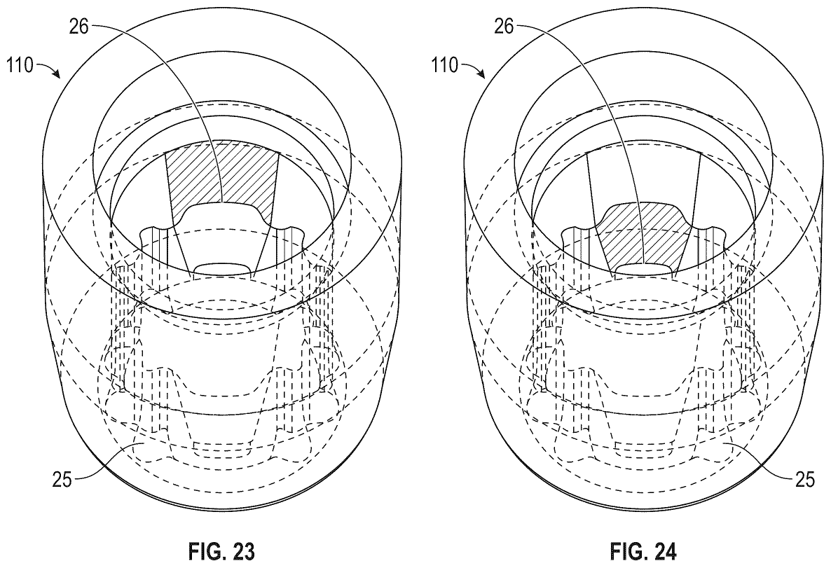

FIG. 23 is a perspective view illustrating a proximal lofted section of a tip of the present disclosure. FIG. 24 is a perspective view illustrating a distal lofted section of a tip of the present disclosure.

As illustrated in FIGS. 23 and 24, a lofted inner transition area 26 is proved at or adjacent to the distal end of the groove, which is provided in an inner surface of a distal end of the channel, which can accommodate an end adaptor. Engaging a protuberance 560 in the lofted inner transition area 26 radially and longitudinally fixes the end adaptor relative to the distal end. The radial fixing of the end adaptor with the distal end of the channel provides an imaging device imaging device integral with or secured by the end adaptor with a predefined field of view. The longitudinal fixing of the end adaptor with the distal end of the channel precludes at least one of walk back of the end adaptor during a flush operation and/or the end adaptor extending from the distal end.

The longitudinal fixing and preclusion of longitudinal movement suppresses walk-back of the inserted image sensor 14 that may occur due to pressure against the end adaptor 500 when a flush operation is performed. Precluding both longitudinal and rotational movement substantially eliminates roll, pitch, yaw and/or walk-back of the end adaptor 500 relative to the continuum robot 100 and improves reliability of a frame of reference between the imaging device and the tip of the continuum robot.

### <Inspection System>

An inspection system is provided that includes a continuum robot 100 with at least one bending section having a distal end and a proximal end, an inner working channel 300 extending through the at least one bending section, and a first groove 320 in an inner surface of the channel 300, the first groove 320 extending from a distal end toward the proximal end. An end adaptor 500 arranged to travel through the channel 300 is provided, with the end adaptor 500 having a first protuberance 560 arranged to engage the first groove 320. Engaging the first protuberance 560 in the first groove 320 precludes independent rotation of the end adaptor 500 relative to the distal end of the at least one bending section of the continuum robot 100.

In the inspection system, the channel 300 and the distal end of the at least one bending section have a substantially circular cross section, and the first groove 320 twists in the substantially circular cross section as the first groove 320 extends from the distal end toward the proximal end.

The continuum robot 100 may include a second groove in the inner surface of the channel 300 extending from the position adjacent to the distal end of the at least one bending section, with the second groove being spaced apart from the first groove by a predetermined number of degrees, and with the first groove and the second groove being equidistantly and/or symmetrically arranged. The first groove and/or the second groove may each provide a lock position.

The continuum robot 100 includes a second groove in the inner surface of the channel 300 extending from the position adjacent to the distal end of the at least one bending section, as well as a third groove in the inner surface of the channel 300, at the distal end of the at least one bending section. The first groove may be spaced apart from the second groove by a predetermined number of degrees, the second groove may be spaced apart from the third groove by the predetermined number of degrees, and the third groove may be spaced apart from the first groove by the predetermined number of degrees. Thus, the first groove, the second groove and the third groove are equidistantly spaced apart and/or symmetrically arranged. The first groove, the second groove and the third groove may each provide a lock position. Symmetrical arrangement of the first groove, the second groove and the third groove may provide a flower-shaped opening at the distal end to the continuum robot 100.

The inspection system may also include an alignment marking provided at a predetermined radial position at or adjacent to a proximal end of the channel. The at least one alignment marking is visible to a user of the inspection system, and the predetermined radial position corresponds to a radial position of the at least one groove.

The inspection system may also include a shaft 13 for moving the end adaptor 500 through the channel 300, with an inner cover 19, a tip of the continuum robot 100, and at least a portion of the shaft 13 being formed of thermoplastic material and being configured to be thermally bonded by reflow and/or split die bonding.

### <Method of Use>

Disclosed are inspection methods that include moving a distal end of a continuum robot 100 to a position at or near a region to be inspected, the continuum robot 100 including a channel 300, a groove 320 and a lofted inner transition area 26. The channel 300 may have a substantially circular inner surface. The lofted inner transition area 26 may be provided in a distal part of the groove 320. The groove 320 may be provided in the substantially circular inner surface and may extend from a distal end of the channel 300 toward a proximal end of the continuum robot 100, with the groove 320 twisting in the substantially circular inner surface as the groove 320 extends from the distal end of the continuum robot 100.

The methods include inserting an end adaptor 500 into a proximal end of the channel 300, the end adaptor 500 including a protuberance 560 having a width that matches a width of the groove 320 and having a profile matching a profile of the lofted inner transition area 26, and moving the end adaptor 500 from the proximal end to a predetermined distance from the distal end of the channel 300. At the predetermined distance, the protuberance 560 engages in the groove 320. Between the predetermined distance and the distal end of the channel 300, the end adaptor 500 continues to move toward the distal end of the channel 300. As the movement continues, the end adaptor 500 rotates by an amount corresponding to the twist of the groove 320. The protuberance 560 then engages in the lofted inner transition area 26 and the inspection may be performed.

Engaging the protuberance 560 in the groove 320 precludes independent rotation of the end adaptor 500 relative to the distal end of the at least one bending section of the continuum robot 100, and engaging the protuberance 560 in the lofted inner transition area 26 rotationally and longitudinally fixes a position of the end adaptor 500. The fixed position aligns a first end 530 of the end adaptor 500 with a tip 110 of the continuum robot 100 and provides a predefined field of view for imaging by an imaging device that may be integral with or secured by the end adaptor 500, without the first end 530 protruding beyond the distal end.

Reference numbers:

| | |
|---|---|
| 2 | Actuator |
| 4, 111, 112, 113 | push/pull drive wires |
| 6 | Hub |
| 11 | Camera access port |
| 12 | Camera can / housing |
| 13 | Shaft |
| 14 | Image sensor |
| 15 | Light source |
| 16 | Drive ring |
| 17 | Guide ring |
| 18 | Anchor |
| 19 | Inner cover |
| 23 | Fin |
| 24 | Tip cutouts |
| 25 | Tip grooves |
| 26 | Lofted inner transition area |
| 27 | Catheter tip proximal bore |
| 28 | Camera distal profile |
| 29 | Proximal camera connector |
| 36 | Fastener |
| 37 | Receiver |
| 38 | Alignment indicator |
| 39 | Proximal aligner |
| 100 | Continuum robot |
| 101 | Display |
| 105 | handheld controller |
| 102, 104, 106 | Bending sections |
| 107 | Tip position sensor |
| 108 | data storage |
| 110 | tip |
| 112 | Flat portion of tip |
| 118 | Rail |
| 121, 122, 123 | Connection portions |
| 160 | End disc |
| 300 | Channel |
| 306 | Sensor |
| 310 | Channel access port |
| 320 | Groove |
| 500 | End adaptor |
| 530 | First end |
| 550 | Body |
| 560 | Protuberance |
| 562 | protuberance distal end |
| 563 | center |
| 564 | Substantially flat portion |
| 565 | substantially flat first wall |
| 566 | substantially flat second wall |
| 568 | proximal part of protuberance |
| 569 | Camera distal profile |
| 570 | Second end |
| P1 | First plane |
| P2 | Second plane |
| LD | Longitudinal direction |
| TD | Transverse direction |
| 1000 | display controller |
| 1106 | EM tracking sensor |

The terms “optical fiber”, “fiber optic”, or simply “fiber” refers to an elongated, flexible, light conducting conduit capable of conducting light from one end to another end due to the effect known as total internal reflection. The terms “light guiding component” or “waveguide” may also refer to, or may have the functionality of, an optical fiber. The term “fiber” may refer to one or more light conducting fibers. An optical fiber has a generally transparent, homogenous core, through which the light is guided, and the core is surrounded by a homogenous cladding. The refraction index of the core is larger than the refraction index of the cladding. Depending on design choice some fibers can have multiple claddings surrounding the core.

Embodiments can be realized by a computer of a system or apparatus that reads out and executes computer executable instructions (e.g., one or more programs) recorded on a storage medium (which may also be referred to more fully as a non-transitory computer-readable storage medium) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., a CPU, micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a RAM, a ROM, a storage of distributed computing systems, an optical disk (such as a compact disc (CD), digital versatile disc (DVD), or Blu-ray Disc (BD)™), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless).

The detector interface also provides communication interfaces to input and output devices. The detector may include, for example a photomultiplier tube (PMT), a photodiode, an avalanche photodiode detector (APD), a charge-coupled device (CCD), multi-pixel photon counters (MPPC), or the like. Also, the function of detector may be realized by computer executable instructions (e.g., one or more programs) recorded on a storage/RAM.

In referring to the description, specific details are set forth in order to provide a thorough understanding of the examples disclosed. In other instances, well-known methods, procedures, components, and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various FIGS. It should be understood, however, that the spatially relative terms are intended to encompass different orientations of the device in use or operation in addition to the orientation depicted in the FIGS. For example, if the device in the FIGS. is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The present disclosure has several embodiments and relies on patents, patent applications and other references for details known to those of the art. Each patent, patent publication and other publication identified herein and examples described herein are expressly incorporated herein by reference. These publications are provided solely for their disclosure prior to the filing date or priority date of the present application. Nothing in this regard shall be construed as an admission against antedating such publication(s) by virtue of prior invention or other applicable reason. All statements as to the date or representation as to content/disclosure of such publication(s) is based on information available and does not constitute any admission as to accuracy of date of disclosure/publication of such publication(s) or content of such publication(s).

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

What is claimed is:

1. An inspection system comprising:
a continuum robot including a distal end and a proximal end, a channel, and a first groove in an inner surface of the channel, the first groove extending from a distal end of the channel toward the proximal end; and
an end adaptor configured to travel through the channel, the end adaptor including a first protuberance configured to engage the first groove,
wherein engaging the first protuberance in the first groove precludes independent rotation of the end adaptor.

2. The inspection system of claim 1, wherein:
the distal end of the channel has a substantially circular cross section, and
the first groove twists in the substantially circular cross section as the first groove extends from the distal end toward the proximal end.

3. The inspection system of claim 1, wherein:
the continuum robot includes at least one second groove in the inner surface of the channel extending from the position adjacent to the distal end of the channel,
the at least one second groove is spaced apart from the first groove by a predetermined number of degrees, and
the first groove and the at least one second groove are equidistantly and/or symmetrically arranged.

4. The inspection system of claim 3, wherein the first groove and/or the at least one second groove provide a lock position.

5. The inspection system of claim 1, further comprising an alignment marking, wherein:
the at least one alignment marking is provided at a predetermined radial position at or adjacent to a proximal end of the channel,
the at least one alignment marking is visible to a user of the inspection system, and the predetermined radial position corresponds to a radial position of the at least one groove.

6. The inspection system of claim 1, further comprising a shaft configured to move the end adaptor through the channel, wherein:
an inner cover, a tip of the continuum robot, and at least a portion of the shaft are formed of thermoplastic material and are configured to be thermally bonded by reflow and/or split die bonding.

7. The inspection system of claim 1, wherein an outer cover of the continuum robot is formed of a thermoplastic material and is configured to be thermally bonded to a tip of the continuum robot.

8. An end adaptor configured to be inserted through a channel that extends through at least a part of a continuum robot, the end adaptor comprising:
a first end;
a second end;
a body extending from the first end to the second end; and
at least one protuberance extending from the body, the at least one protuberance including a distal end, a proximal end, and a center extending between the distal end to the proximal end, wherein:
the second end is configured to abut a shaft configured to move the end adaptor in the channel, and
upon moving the end adaptor to a distal end of the channel, the first end aligns with a tip of the continuum robot and the end adaptor precludes the first end from protruding from the distal end.

9. The end adaptor of claim 8, wherein aligning the first end with the tip provides a predefined field of view to perform imaging by an imaging device that is integral with or secured by the end adaptor.

10. The end adaptor of claim 8, wherein the center includes a substantially flat portion corresponding to a predetermined position of an imaging device to be secured by the end adaptor.

11. The end adaptor of claim 8, wherein the at least one protuberance is configured to engage a groove in an inner surface of the channel, the groove extending from a position adjacent to a distal end of the continuum robot.

12. The end adaptor of claim 11, wherein the engaging precludes independent rotation of the end adaptor.

13. The end adaptor of claim 8, wherein the distal end and the proximal end include respective substantially concave regions between a first plane and a substantially parallel second plane, with the first plane extending along an exterior of the body at a lower end of the at least one protuberance and the second plane extending along an apex of the center.

14. A continuum robot comprising:
a channel; and
a groove provided in an inner surface of a distal end of the channel, wherein:
the groove includes a lofted area at or adjacent to the distal end, and
the channel is configured to accommodate an end adaptor.

15. The continuum robot of claim 14, further comprising at least one alignment marking provided at a predetermined radial position at or adjacent to a proximal end of the channel, wherein the predetermined radial position aligns with the groove.

16. The continuum robot of claim 14, wherein:
the end adaptor includes a protuberance, and
moving the end adaptor through the channel to the distal end engages the protuberance in the lofted area.

17. The continuum robot of claim 16, wherein the engaging of the protuberance in the lofted area radially and longitudinally fixes the end adaptor relative to the distal end.

18. The continuum robot of claim 16, wherein the engaging of the protuberance in the lofted area radially fixes the end adaptor with the distal end of the channel to provide an imaging device imaging device integral with or secured by the end adaptor with a predefined field of view.

19. The continuum robot of claim 16, wherein:
a distal end of the end adaptor is substantially rectangular shaped, and
when the end adaptor is inserted into a tip of the continuum robot, a clearance is formed between the channel and the substantially rectangular shaped distal end of the end adaptor to allow fluid flow through the channel with the protuberance engaged in the lofted area.

20. The continuum robot of claim 16, wherein the engaging of the protuberance in the lofted area longitudinally fixes the end adaptor with the distal end of the channel.

21. The continuum robot of claim 20, wherein the longitudinal fixing precludes at least one of walk back of the end adaptor and/or the end adaptor extending from the distal end.

22. An inspection method comprising:
moving a distal end of a continuum robot to a position at or near a region to be inspected, the continuum robot including a channel, a groove and a lofted area, with:
the channel having a substantially circular inner surface,
the lofted area being provided in a distal part of the groove, and
the groove being provided in the substantially circular inner surface and extending from a distal end of the channel toward a proximal end of the continuum robot, with the groove twisting in the substantially circular inner surface, as the groove extends from the distal end of the continuum robot;
inserting an end adaptor into a proximal end of the channel, the end adaptor including a protuberance having a width matching a width of the groove and having a profile matching a profile of the lofted area;
moving the end adaptor from the proximal end to a predetermined distance from the distal end of the channel;
at the predetermined distance, engaging the protuberance in the groove;
between the predetermined distance and the distal end of the channel, continuing to move the end adaptor toward the distal end of the channel, with the continued movement rotating the end adaptor corresponding to the twist of the groove;
engaging the protuberance in the lofted area; and
performing the inspection.

23. The inspection method of claim 22, wherein the engaging of the protuberance in the groove precludes independent rotation of the end adaptor.

24. The inspection method of claim 22, wherein the engaging of the protuberance in the lofted area rotationally and longitudinally fixes a position of the end adaptor.

25. The inspection method of claim 24, wherein, when in the fixed position, a distal end of the end adaptor aligns with a tip of the continuum robot to provide a predefined field of view for an imaging device integral with or secured by the end adaptor, without the distal end of the end adaptor protruding beyond the distal end of the channel.
